(19) United States
(12) Patent Application Publication
(10) Pub. No.: US 2026/0116978 A1
(43) Pub. Date: Apr. 30, 2026

(54) Methods of Performing Boron Neutron Capture Therapy (BNCT) in Combination with Immune Checkpoint Inhibitors to Enhance BNCT and the Anti-tumor effects of Immune Check Point Inhibitors as well as the Abscopal Effect Induced by Radiation Therapy and Methods Thereof

(71) Applicant: TAE Life Sciences LLC, Irvine, CA (US)

(72) Inventors: Dr. Robert Kendall Morrison, Santa Monica, CA (US); Dr. Arthur B. Raitano, Los Angeles, CA (US)

(21) Appl. No.: 19/371,705

(22) Filed: Oct. 28, 2025

Related U.S. Application Data
(60) Provisional application No. 63/712,685, filed on Oct. 28, 2024.

(51) Int. Cl. C07K 16/28; A61K 9/00; A61K 39/00; A61K 41/00; A61P 35/00

(52) U.S. Cl. CPC C07K 16/2818; A61K 9/0019; A61K 41/0095; A61P 35/00; C07K 16/2827; A61K 2039/505

(57) ABSTRACT

Disclosed herein is a treatment method utilizing Boron Neutron Capture Therapy (BNCT) in combination with immune checkpoint inhibitors (ICIs), such as anti PD1, PDL1 and CTLA4 antibodies. The treatment combination of BNCT with immune check point inhibitors enhances tumor cell recognition by immune cells and stimulate a stronger abscopal effect than is seen with either therapy individually. This synergy provides an enhanced method of treating cancer, immunological disorders and other disease.

### CROSS-REFERENCE TO RELATED APPLICATIONS

This application claims priority to U.S. Provisional Patent Application No. 63/712,685 filed 28 Oct. 2024, the contents of which are fully incorporated by reference herein.

### STATEMENT OF RIGHTS TO INVENTIONS MADE UNDER FEDERALLY SPONSORED RESEARCH

Not applicable.

### FIELD OF THE INVENTION

The invention described here relates to the field of boron neutron capture therapy (BNCT) and its combination with immune check-point inhibitors (CPH) also known as (Check Point Blockade (CPB). It is well documented that immune system stimulation can be triggered by radiotherapies such as those disclosed herein. This immune cell stimulation is caused by damage to tumor cels and can not only affect the tumor being directly treated with radiotherapy, but also tumors, such as metastases, distant from the original site of direct tumor treatment. This is known as the abscopal effect and is caused by stimulated immune cells such as macrophages, natural killer cells and T-cells, migration through the lymphatic system and recognizing the same tumor type at another distant site.

This invention specifically relates to combination therapy between BNCT and CPB and the subsequent enhancement of both BNCT and CPB in addition to stimulation of the abscopal effect. To achieve this the check point inhibitors can be given before BNCT treatment, during BNCT treatment or after BNCT treatment or a combination of all three. The invention further relates to the treatment of cancers and some non-oncological diseases such as immunological disorders.

### BACKGROUND OF THE INVENTION

External beam radiation therapy is used to treat many kinds of cancers, the second leading cause of death in humans worldwide next to coronary disease. Almost 10 million people die from cancer every year (GLOBOCAN 2022) and in the United States alone cancer kills well over a half-million people annually, with over 1.2 million new cases diagnosed per year (American Cancer Society). Radiotherapy is one of the three pillars of cancer treatment along with chemotherapy and surgery. Although unpredictable, as mechanisms are not fully understood, radiotherapy has long been associated with immune system stimulation as the body reacts to treatment and clears the cell debris at the tumor site caused by tumor cell death. This local destruction of cancer cells at the treatment site, which are mostly cleared by macrophages, causes infiltration of immune cells and can stimulate an immune cell effect known as the abscopal effect (ab-“away” scopus-“target”). This is when local radiation treatment leads to the shrinkage of tumors at untreated sites, particularly local and distant metastases. It was first described in 1953 by RH Mole in the British Journal of Radiology (See, MOLE, RH Brit. J. Radiol., vol. 26, pp. 234-241 (1953). However, since then there have been varying reports of how predictable the abscopal effect is in the intervening seventy (70) years, with substantial variation in patient responses it would be beneficial to find a way that not only combines the effects of BNCT and ICP treatment but also enhances the abscopal effect making it more robust and predictable.

To try and understand it better the number of studies involving the abscopal effect have increased significantly in the last decade. Most recently SCHWINT, et. al., published literature showing promising pre-clinical results associating the abscopal effect with BNCT in animal models. See, TRIVILLIN, et. al., Brit. J. Radil., vol. 94, issue 1128 (2021). If responses in preclinical models would translate to the clinic then a combination between BNCT and check point blockade (CPB) may give more options not only for rarer difficult to trat cancers but also frequently diagnosed cancers such as breast and lung cancer, the highest abundance cancers in women and men respectively for both frequency of occurrence and death.

On this night, deaths from certain types of cancer have been increasing over the past few years and it is expected to continue to increase in subsequent years. From the aforementioned, it is apparent either in the field of cancer research, or other medical fields, that new cancer treatments options are needed.

One such option is BNCT, which is approved for treating recurring head and neck cancer in Japan and is the subject of several other clinical trials throughout the world including Europe, China, Taiwan, Korea as well as Japan. Briefly, Neutron Capture Therapy (NCT), which includes BNCT, is a form of radiation therapy as previously stated. It is based on targeting tumors with drugs that deliver boron preferentially to the tumor cells and not to normal cells giving a biochemical tumor targeting modality. Once boron has been allowed to accumulate in tumor cells (normally after 2 hours) the tumor is irradiated with neutrons generated either in a nuclear reactor or, more recently, from an accelerator-based neutron source. This irradiation of boron enriched tumors with a shower of neutrons caused the release of high energy alpha particles that kill the tumor cells while sparing normal tissue around the tumor. Thus, BNCT uses a dual targeting modality, one from biochemical targeting and the other from the location of the neutron beam relative to the tumor. The fact that treatment with BNCT can stimulate the immune system and give rise to an abscopal effect that affects growth of tumors at distant sites is very important for improved outcomes for cancer treatments.

Although the immune stimulation and abscopal effects induced by BNCT in pre-clinical models are quite strong, both effects may be improved even further by combining BNCT with immune check point inhibitors. ICPs are known to stimulate the immune system and render tumors, which were previously overlooked by immune surveillance, visible to the highly effective family of immune effector cells.

Treatment with immune checkpoint inhibitors such as anti-CTLA4/B7-1 and anti PD-1/PDL1 is one of the fastest growing fields of cancer treatment today. In order to evade the hosts immune system many cancers have adapted to produce signals that render them unrecognizable to the immune system since these cells, such as immune T-cells, and some cancer cells produce check-points that renders a weaker immune response thus preventing tumor cell death and elimination. For example, PD-1, found on T cells, binds to its ligands, PD-L1 and PD-L2, found on many tumor cells, to suppress immune activity and tumor cell killing. As BNCT stimulates the immune system as does the use of CPB, their combination is synergistic, enhancing tumor cell killing and stimulating the abscopal effect, the effect in pre-clinical models has been shown to be devastating to both local and distant tumors.

The idea of combining radiotherapy with immune-checkpoint inhibitors is a combination of the two modalities with the main focus of directly killing tumor cells and killing them indirectly through the stimulation of immunes cells such T-cells. See, MCLAUGHLIN, et. al., Nat. Rev. Cancer 20, 203-217 (2020). Notably, recent studies have shown that combining radiotherapy with activation of macrophages yields potent abscopal effects in mouse tumor models. See, BARKER, et. al., Nat. Cancer 3, pp. 1282-1283 (2022). However, combining BNCT, a sub-type of radiotherapy, with immune checkpoint inhibitors is a novel approach described herein.

From the aforementioned, it will be obvious to those skilled in the art that this combination of BNCT with immune check-point inhibitors offers a viable advantage and effective new treatment of cancers and other non-oncological diseases.

### SUMMARY OF THE INVENTION

In one embodiment the invention provides for neutron capture compositions and methods of performing BNCT comprising a combination of the administration of immune check-point inhibitors as a potent treatment modality to treat human diseases, such as cancer, as well as other disorders such as inflammatory disease, including but not limited to, rheumatoid arthritis, Alzheimer's disease, ankylosing spondylitis, and other cellular diseases. In certain embodiments, this combination therapy consists of the combination of BNCT with immune check-point inhibitors such as (i) KEYTRUDA (pembrolizumab), (ii) OPDIVO (nivolumab), (iii) YERVOY (ipulimumab), and (iv) TECENTRIQ (atezolizumab), four (4) of several drugs approved by the FDA for immunotherapy. Others include LIBTAYO (cemiplimab), JEMPERELI (dostarlimab) and TEVIMBRA (tislelizumab) all PD-1 inhibitors; IMFINZI (durvalumab), BEVENCIO (avelumab), PD-L1 inhibitors and IMJUDO (tremelimumab), all CTLA4 inhibitors. More recently, OPDUALAG (relatlimab) was also approved, this targets LAG-3. As listed, the main check point inhibitors target either PD-1 PD-L1, CTLA4 or LAG3 and the field has expanded rapidly so that some of the drugs listed above are generic versions of previously approved drugs whose patents have expired. These antibody-based biologics are able to switch off immune check point inhibitors and when used in conjunction with BNCT hyper-stimulate the immune system to eradicate the cancer at locally treated and distant sites. There are multiple clinical trials ongoing combining CPB with other established treatments thus endorsing the idea that these drugs can be used in combination with other treatments.

In a further embodiment, the invention comprises methods of concentrating Boron in a cell comprising (i) synthesizing a borylated di-peptide composition, (ii) administering the borylated di-peptide to a patient, (iii) irradiating the cell with neutrons produced in a neutron source, (iv) administering an immune checkpoint inhibitor before, on day of or after BNCT or a combination of these three check point inhibitor administration approaches.

In a further embodiment, the invention comprises methods of concentrating Boron in a cell comprising (i) synthesizing a borylated amino acid (“BAA”) composition, (ii) administering the BAA to a patient, (iii) irradiating the cell with neutrons produced in a neutron source, (iv) administering an immune checkpoint inhibitor before, on day of or after BNCT or a combination of these three check point inhibitor administration approaches

In a further embodiment the invention comprises methods of concentrating Boron in a cell comprising (i) synthesizing a BEL; conjugating a BEL of the invention to an antibody, creating an antibody boron conjugate (ABC); (ii) administering the ABC to a patient, (iii) irradiating the cell with neutrons produced in a neutron source, (iv)
administering an immune checkpoint inhibitor before, on day of or after BNCT or a combination of these three-check point inhibitor administration approaches.

In a further embodiment, the invention comprises methods of concentrating Boron in a cell comprising (i) synthesizing a boronated carbohydrate (BC) composition, (ii) administering the BC to a patient, (iii) irradiating the cell with neutrons produced in a neutron source, (iv) administering an immune checkpoint inhibitor before, on day of or after BNCT or a combination of these three check point inhibitor administration approaches.

In a further embodiment, the invention comprises methods of concentrating Boron in a cell comprising (i) synthesizing a borylated di-peptide composition, (ii) administering the borylated di-peptide to a patient, (iii) irradiating the cell with neutrons produced in a neutron source, (iv) administering an immune checkpoint inhibitor.

In a further embodiment, the invention comprises methods of concentrating Boron in a cell comprising (i) synthesizing a borylated amino acid (“BAA”) composition, (ii) administering the BAA to a patient, (iii) irradiating the cell with neutrons produced in a neutron source, (iv) administering an immune checkpoint inhibitor.

In another embodiment, the present disclosure teaches a treatment paradigm comprising BNCT and an immune checkpoint inhibitor.

In another embodiment, the present disclosure teaches a treatment paradigm comprising BNCT and an immune checkpoint inhibitor comprising KEYTRUDA.

In another embodiment, the present disclosure teaches a treatment paradigm comprising BNCT and an immune checkpoint inhibitor comprising OPDIVO.

In another embodiment, the present disclosure teaches a treatment paradigm comprising BNCT and an immune checkpoint inhibitor comprising YERVOY.

In another embodiment, the present disclosure teaches a treatment paradigm comprising BNCT and an immune checkpoint inhibitor comprising TENCENTRIQ.

In another embodiment, the present disclosure teaches a treatment paradigm comprising BNCT and an immune checkpoint inhibitor comprising LIBTAYO (cemiplimab).

In another embodiment, the present disclosure teaches a treatment paradigm comprising BNCT and an immune checkpoint inhibitor comprising JEMPERELI (dostarlimab).

In another embodiment, the present disclosure teaches a treatment paradigm comprising BNCT and an immune checkpoint inhibitor comprising TEVIMBRA (tislelizumab).

In another embodiment, the present disclosure teaches a treatment paradigm comprising BNCT and an immune checkpoint inhibitor comprising IMFINZI (durvalumab).

In another embodiment, the present disclosure teaches a treatment paradigm comprising BNCT and an immune checkpoint inhibitor comprising BEVENCIO (avelumab).

In another embodiment, the present disclosure teaches a treatment paradigm comprising BNCT and an immune checkpoint inhibitor comprising IMJUDO (tremelimumab).

In another embodiment, the present disclosure teaches a treatment paradigm comprising BNCT and an immune checkpoint inhibitor comprising OPDUALAG (relatlimab).

In another embodiment, the present disclosure teaches a treatment paradigm comprising BNCT and an immune checkpoint inhibitor comprising and anti-mPD1 antibody.

In another embodiment, the present disclosure teaches a treatment paradigm comprising BNCT and an immune checkpoint inhibitor comprising and anti-PD1 antibody.

In another embodiment, the present disclosure teaches a treatment paradigm comprising BNCT and an immune checkpoint inhibitor comprising and anti-PD-L1 antibody.

In another embodiment, the present disclosure teaches a treatment paradigm comprising BNCT and an immune checkpoint inhibitor comprising and anti-CTLA-4 antibody.

In another embodiment, the present disclosure teaches a treatment paradigm comprising BNCT and an immune checkpoint inhibitor comprising an anti-LAG3 antibody.

In another embodiment, the present disclosure teaches a treatment paradigm comprising BNCT and an immune checkpoint inhibitor, wherein the BNCT modality comprises a neutron capture agent comprising BTS.

In another embodiment, the present disclosure teaches a treatment paradigm comprising BNCT and an immune checkpoint inhibitor, wherein the BNCT modality comprises a neutron capture agent comprising BPA-BPA.

In another embodiment, the present disclosure teaches a treatment paradigm comprising BNCT and an immune checkpoint inhibitor, wherein the BNCT modality comprises a neutron capture agent comprising antibody boron conjugate (ABC).

In another embodiment, the present disclosure teaches a treatment paradigm comprising BNCT and an immune checkpoint inhibitor, wherein the BNCT modality comprises a neutron capture agent comprising a boronated GLUT-1 transporter.

In another embodiment, the present disclosure teaches a treatment paradigm comprising BNCT and an immune checkpoint inhibitor, wherein the BNCT modality comprises a neutron capture agent comprising a carborane boron enriched linker (BEL).

In another embodiment, the present disclosure teaches methods of treating cancer(s), immunological disorders, and other diseases in humans.

### BRIEF DESCRIPTION OF THE FIGURES

FIG. 1. Schematic Representation of Protocol.

FIG. 2. Mechanism of the Abscopal Effect at the Cellular Level.

FIG. 3. Mechanism of Immune Checkpoint Inhibitor(s).

FIG. 4. Experimental Design to Generate Abscopal Effect(s) in Animal Models.

FIG. 5. Abscopal Effect in BNCT Mouse Allogenic Model, In vivo. FIG. 5(A). Shows the test of abscopal/vaccine effect using ¹⁰BPA-BPA and ¹⁰Ala-BPA. FIG. 5(B). Shows the test of abscopal/vaccine effect using ¹⁰BPA-BPA and ¹⁰Ala-BPA without the outlier.

FIG. 6. BNCT with Dipeptide ¹⁰BPA-BPA Inhibits CT26 Tumors Causing Immune Cell Infiltrates.

FIG. 7. Induction of Abscopal Effect by BNCT Using BPA-BPA, In vivo. FIG. 7(A). Shows the tumor inhibition on the primary irradiated tumor with ¹⁰BPA-BPA versus ¹⁰BPA. FIG. 7(B). Shows the test of abscopal/vaccine effect after implantation following BNCT with 19BPA-BPA versus ¹⁰BPA.

FIG. 8. Induction of Abscopal Effect by BNCT Using BTS, In vivo. FIG. 8(A). Shows the tumor inhibition on the primary irradiated tumor with ¹⁰BTS. FIG. 8(B). Shows the test of abscopal/vaccine effect after implantation following BNCT with ¹⁰BTS.

FIG. 9. Induction of abscopal effect by BNCT Using BPA-BPA in combination with PD-1 Antibody at Multiple Doses, in vivo.

FIG. 10. Day Eight (8) Evaluation of Induction of abscopal effect by BNCT Using BPA-BPA in combination with PD-1 Antibody at Multiple Doses, in vivo. FIG. 10(A). Shows the day eight (8) snapshot of the tumor inhibition on the primary irradiated tumor. FIG. 10(B). Shows the total days post-treatment of the tumor inhibition on the primary tumor.

FIG. 11. Day Eight (8) Evaluation of Distal (Abscopal) Tumor versus Irradiated Tumor. FIG. 11(A). Shows day eight (8) snapshot of the distal tumor (abscopal tumor) in the left shoulder. FIG. 11(B). Shows day eight (8) snapshot of the irradiated tumor in the right leg.

FIG. 12. Day Sixteen (16) Evaluation of Distal (Abscopal) Tumor versus Irradiated Tumor. FIG. 12(A). Shows the day sixteen (16) snapshot of the distal tumor (abscopal tumor) in the left shoulder. FIG. 12(B). Shows day sixteen (16) snapshot of the irradiated tumor in the right leg.

FIG. 13. Day Zero (0) Versus Sixteen (16) Evaluation of Distal (Abscopal) Tumor versus Irradiated Tumor. FIG. 13(A). Shows day zero (0) snapshot of the distal tumor (abscopal tumor) in the left shoulder. FIG. 13(B). Shows the day sixteen (16) snapshot of the distal tumor (abscopal tumor) in the left shoulder. FIG. 13(C). Shows day zero (0) snapshot of the primary tumor in the right leg. FIG. 13(D). Shows day sixteen (16) snapshot of the primary tumor in the right leg.

FIG. 14. Day Thirteen (13) Evaluation of Irradiated Tumor (Right Leg) versus Distal (Abscopal) Tumor (Left Shoulder). FIG. 14(A). Shows the day thirteen (13) snapshot of the irradiated tumor (right leg). FIG. 14(B). Shows the day thirteen (13) snapshot of the distal tumor (abscopal tumor) in the left shoulder. FIG. 14, continued, Day Sixteen (16) Evaluation of Irradiated Tumor (Right Leg) versus Distal (Abscopal) Tumor (Left Shoulder). FIG. 14(C). Shows day sixteen (16) snapshot of the irradiated tumor (right leg). FIG. 14(D). Shows the day sixteen (16) snapshot of the distal tumor (abscopal tumor) in the left shoulder. FIG. 14, continued, Day Twenty-Four (24) Evaluation of Irradiated Tumor (Right Leg) versus Distal (Abscopal) Tumor (Left Shoulder). FIG. 24(E). Shows the day twenty-four (24) snapshot of the irradiated tumor (right leg). FIG. 14(F). Shows the day twenty-four (24) snapshot of the distal tumor (abscopal tumor) in the left shoulder.

### DETAILED DESCRIPTION OF THE INVENTION

### Outline of Sections

I.) Definitions
II.) Neutron Capture Therapy (NCT) and Boron Neutron Capture Therapy (BNCT)
III.) Abscopal Effect
IV.) Immune Checkpoint Inhibitors
   a. Keytruda
   b. Opdivo
   c. Yervoy
   d. Tencentriq
V.) Immune Checkpoint Specific Antibodies
VI.) Clinical Design for Immune Checkpoint Inhibitors with BNCT
VII.) Methods of Treating Cancer(s) and Other Immunological Disorder(s)
   a. Combination Therapy
IX.) KITS/Articles of Manufacture

### 1.) Definitions

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

The terms “advanced cancer”, “locally advanced cancer”, “advanced disease” and “locally advanced disease” mean cancers that have extended through the relevant tissue capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) cancer.

The term “antibody” is used in the broadest sense unless clearly indicated otherwise. Therefore, an “antibody” can be naturally occurring or man-made such as monoclonal antibodies produced by conventional hybridoma technology. Furthermore, antibodies comprise monoclonal and polyclonal antibodies as well as fragments containing the antigen-binding domain and/or one or more complementarity determining regions of these antibodies. As used herein, the term “antibody” refers to any form of antibody or fragment thereof that specifically binds a target antigen and/or exhibits the desired biological activity and specifically covers monoclonal antibodies (including full length monoclonal antibodies), polyclonal antibodies, Mult specific antibodies (e.g., bispecific antibodies), and antibody fragments so long as they specifically bind a target antigen or fragment thereof and/or exhibit the desired biological activity. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. In one embodiment, the antibody is an IgG antibody. For example, the antibody is an IgG1, IgG2, IgG3, or IgG4 antibody. The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, and apes. Therefore, in one embodiment, an antibody of the present invention is a mammalian antibody. Phage techniques can also be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, ANTIBODY PRODUCTION: ESSENTIAL TECHNIQUES (Wiley, 1997); Shephard, et al., MONOCLONAL ANTIBODIES (Oxford University Press, 2000); Goding, MONOCLONAL ANTIBODIES: PRINCIPLES AND PRACTICE (Academic Press, 1993); and CURRENT PROTOCOLS IN IMMUNOLOGY (John Wiley & Sons, most recent edition).

An antibody of the present invention can be modified by recombinant means to increase efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to normal or defective 158P1D7. See e.g., ANTIBODY ENGINEERING: A PRACTICAL APPROACH (Oxford University Press, 1996). Suitable antibodies with the desired biologic activities can be identified using the following in vitro assays including but not limited to: proliferation, migration, adhesion, soft agar growth, angiogenesis, cell-cell communication, apoptosis, transport, signal transduction, and the following in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

The term “antigen-binding portion” or “antibody fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)² fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarily determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, any form of the “antigen” can be used to generate an antibody that is specific for the target. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may be genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least a portion of the extracellular domain of the target of interest and fragments thereof.

“Antibody Boron Conjugate (“ABC”) is an important class of biopharmaceutical drugs designed as a targeted therapy to enhance Boron Neutron Capture Therapy (BNCT). Unlike ADCs, which consist of antibodies combined with a toxic payload, ABCs are made up of an antibody, or antibody fragment, conjugated with a non-cytotoxic, boron containing molecule such as a Boron Enriched Linker (BEL). It is not until the boron in the ABC is irradiated with epithermal neutrons in a BNCT treatment that it releases a cell killing alpha particle. This type of treatment is currently used in cancer treatment and may also be a suitable for other disease indications. In contrast to chemotherapy and ADC treatment, targeted BNCT using ABCs has the potential to kill only the cancer cells and spare healthy cells. Antibody Boron Conjugates are examples of bioconjugates and immunoconjugates.

“Bispecific” antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The terms “inhibit” or “inhibition of” as used herein means to reduce by a measurable amount, or to prevent entirely.

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “metastatic cancer” and “metastatic disease” mean cancers that have spread to regional lymph nodes or to distant sites and are meant to include stage D disease under the AUA system and stage T×N×M+ under the TNM system.

“Pharmaceutically acceptable” refers to a non-toxic, inert, and/or composition that is physiologically compatible with humans or other mammals.

The term “neutron capture agent” means a stable non-radio reactive chemical isotope which, when activated by neutrons produces alpha-rays and gamma-rays.

The term “neutron capture therapy” means a noninvasive therapeutic modality for treating locally invasive malignant tumors such as primary brain tumors and recurrent head and neck cancer and other immunological disorders and disease by irradiating a neutron capture agent with neutrons.

As used herein, the terms “specific,” “specifically binds” and “binds specifically” refer to the selective binding of the antibody to the target antigen epitope. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific. In one embodiment, a specific antibody is one that only binds the target antigen but does not bind to the irrelevant antigen. In another embodiment, a specific antibody is one that binds a human target antigen but does not bind a non-human target antigen with 70%, 75%, 80%, 85%, 90%, 91%, 92%, 93%, 94%, 95%, 96%, 97%, 98%, 99% or greater amino acid homology with the human target antigen. In another embodiment, a specific antibody is one that binds a human target antigen and binds a murine target antigen, but with a higher degree of binding the human antigen. In another embodiment, a specific antibody is one that binds a human target antigen and binds a primate target antigen, but with a higher degree of binding the human target antigen. In another embodiment, the specific antibody binds to a human target antigen and any non-human target antigen, but with a higher degree of binding the human antigen or any combination thereof.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

As used herein the term “vaccine effect” means an occurrence when immune memory leads to growth inhibition on re-challenge with the same organism or tumor.

As used herein the term “abscopal effect” means an occurrence when local radiation treatment leads to shrinkage of tumors at distant, untreated sites. As used herein, the abscopal effect can occur to metastasis or micro-metastasis.

As used herein the term “immune checkpoint inhibitor (ICI)” refers to, in its broadest sense, means a type of drug that blocks proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better. Non-limiting examples of checkpoint proteins found on T cells or cancer cells include but are not limited to PD-1/PD-L1 and CTLA-4/B7-1/B7-2.

### II.) Neutron Capture Therapy (NCT) and Boron Neutron Capture Therapy (BNCT)

Neutron Capture Therapy (NCT) is a promising form of radiation therapy. BNCT, which is a type of NCT, is a technique that selectively kills tumor cells using a targeted boron compound that selectively accumulates in tumor cells while sparing the normal cells. BNCT relies on the propensity of non-radioactive ¹⁰B isotope to absorb epithermal neutrons that fall into the low energy range of 0.5 keV<Eₙ<30 keV. Following neutron capture, the boron atom undergoes a nuclear fission reaction giving rise to an alpha-particle and a recoiled lithium nucleus (⁷Li) as follows:

$${}^{10} B + n \to {}^{7} Li + {}^{4} He$$

The alpha particle deposits high energy i.e., 150 keV/μm along their short path essentially restricted to a single cell diameter that results in a double strand DNA breaks followed by cancer cell death by apoptosis. Thus, BNCT integrates a concept of both chemotherapy, targeted therapy, and the gross anatomical localization of traditional radiotherapy.

Carriers of boron have evolved since 1950s and are reviewed in NEDUNCHEZHIAN, et. al., J. Clin. Diag. Res., vol. 10 (12) (December 2016). Briefly, the 1^{st} generations of boron compounds represented by boric acid and its derivatives were either toxic or suffered from low tumor accumulation/retention. BPA and BSH are both considered the 2^{nd} generation compounds that emerged in 1960s. These had significantly lower toxicity and better PK and biodistribution. BPA-fructose complex is considered the 3^{rd} generation compound that is used to treat patients with H&N, glioblastoma and melanoma using BNCT since 1994. BPA-fructose and BSH are the only compounds that are being used in clinic as boron carriers to date although both low and high molecular weight biomolecules such as nucleosides, porphyrins, liposomes, nanoparticles and mAbs have been evaluated for the tumor targeting in preclinical models. The main deficiency of BPA-fructose is relatively low solubility combined with its rapid clearance that prevents achieving high Cmax in blood, one of the drivers influencing the tumor uptake.

Even though the conceptual techniques of NCT and specifically Boron Neutron Capture Therapy (BNCT) are well known, the technological limitations associated with this type of treatment have slowed progress. During the early investigations using the research reactors of MIT in 1960's, several dozens of patients were treated using disodium decahydrodecaborate, which was considered less toxic than simple boron compounds used previously yet capable of delivering more boron to the cell. Unfortunately, disodium borocaptate use did not improve outcomes and BNCT studies were halted in the USA. The primary dose limiting toxicity observed was severe brain necrosis in the patients undergoing BNCT.

Hiroshi Hatanaka in 1968 re-investigated clinical application of BNCT in Japan using sodium borocaptate (BSH) by directing the beam to surgically exposed intracranial tumor and reported of achieving 58% of 5-year survival rate. In 1987 clinicians in Japan applied BNCT for the treatment of malignant melanoma using boronophenylalanine (BPA) as boron compound. Thus, slow resurgence of BNCT took place albeit limited to the countries with access to research reactor facilities capable of delivering epithermal neutron beam. Currently, given the technological improvements in both (i) the infusion and delivery of a capture compound, which preferably concentrates in the tumor, and (ii) more abundant and easier access to neutron beam using cyclotrons, there has been a resurgence in NCT treatment methods.

One aspect of the present disclosure is the use of borylated amino acids as alternatives to BPA such as BTS and/or BTS(OMe) (TAE Life Sciences, LLC, Santa Monica, CA) as a modality for Boron Neutron Capture Therapy (“BNCT”).

Another aspect of the present disclosure is the use of borylated di-peptide composition(s) (e.g., ¹⁰BPA-BPA and/or ¹⁰Ala-BPA) (TAE Life Sciences, LLC, Santa Monica, CA) as a modality for Boron Neutron Capture Therapy (“BNCT”).

Given that BNCT is usually a single treatment (unlike most forms of radiation therapy that are often fractioned over 30 fractions or more, BNCT is much more tolerable for patients and hospital staff alike. The other advantage is that BNCT can be a successful treatment without a highly traumatic surgical procedure or debilitating rounds of chemotherapy. However, as will be understood by one of skill in the art, success is predicated by high concentration and selective localization of ¹⁰B in tumor cells and can be enhanced even more when used in combination with immune check point inhibitors.

In the aforementioned embodiment, one aspect of the present disclosure is the treatment of disease using BNCT in combination with an immune checkpoint inhibitor.

### III.) Abscopal Effect

Generally speaking, The abscopal effect occurs when radiation treatment—or another type of local therapy—not only affects the targeted tumor but also has an inhibitory effect on untreated tumors elsewhere in the body. The abscopal effect is thought to occur when local therapy stimulates the immune system to causing it to recognize tumor cells throughout the body. The immune system may recognize tumor cells as a threat after radiation treatment releases material from the tumor cells that are recognized as foreign. Initially associated with radiation therapy, the term “abscopal effect” has also come to encompass other types of localized treatments such as cryo-ablation, electroporation and intra-tumoral injection of therapeutics. However, it is notable that the term should only be used when truly local treatments result in systemic effects. This is because, as in most systemic therapies, there are instances where active drugs circulate through the bloodstream but due to their intrinsic activity exclude the possibility of any abscopal response. This is the case with chemotherapeutics and antibody therapies such as immune checkpoint inhibitors.

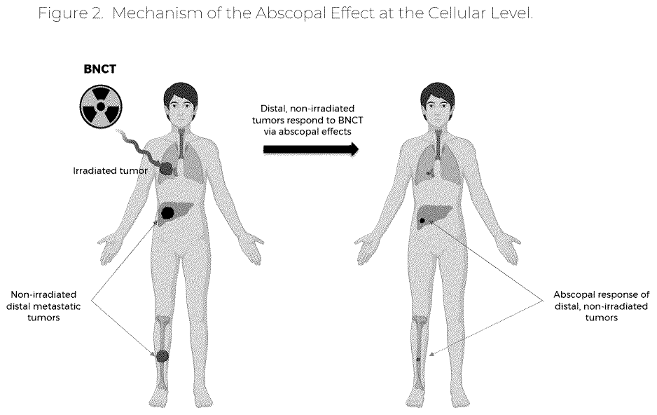

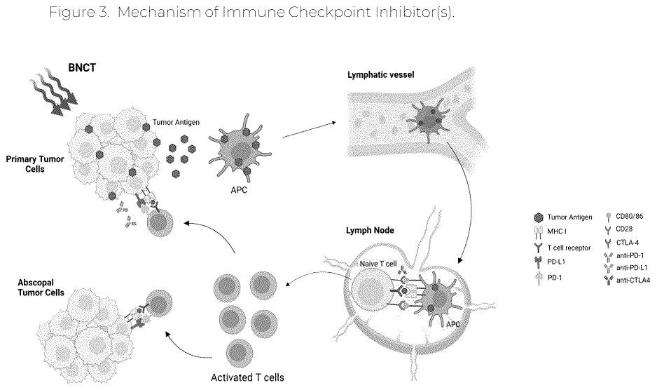

Similar to immune reactions against antigens from bacteria or viruses, the abscopal effect requires priming of immune cells against tumor antigens. Local irradiation of a tumor may lead to immunogenic forms of tumor cell death and liberation of tumor cell-derived antigens. These antigens can be recognized and processed by antigen-presenting cells within the tumor (dendritic cells and macrophages). Cytotoxic T cells, which recognize these tumor antigens, may in turn be activated by the tumor antigen-presenting cells. In contrast to the local effect of irradiation on the tumor cells, these cytotoxic T cells circulate through the blood stream (See, FIG. 2 and FIG. 3) and are thus able to destroy remaining tumor cells in distant parts of the body which were not irradiated. Accordingly, increases in tumor-specific cytotoxic T cells have been shown to correlate with abscopal anti-tumor responses in patients. Vice versa, the abscopal effect is abolished after experimental depletion of T cells in various animal models.

Finally, abscopal effects of ionizing radiation are often blocked by the immunosuppressive microenvironment inside the irradiated tumor which prevents effective T cell activation. This explains why the effect is rarely seen in patients receiving radiotherapy alone. In contrast, the combination with immunomodulatory drugs such as ipilimumab and pembrolizumab can partially reconstitute systemic anti-tumor immune reactions induced after local tumor radiotherapy. The optimal combination of radiation dose and combination with immunomodulatory drugs is currently under intensive investigation along with multiple other clinical trials combining immunomodulatory drugs with other established therapies. In this context, it was proposed that radiation doses above 10 to 12 Gray might be ineffective in inducing immunogenic forms of cell death. However, there is so far no consensus on the optimal radiation regimen needed to increase the chance of abscopal tumor regression.

Moreover, studying the abscopal effect in the context of BNCT is a novel treatment paradigm which is more thoroughly disclosed herein.

### IV.) Immune Checkpoint Inhibitors

Generally speaking, and for the purposes of this disclosure, immune checkpoint inhibitors are a class of drug(s) that block proteins called immune checkpoints that are made by some types of immune system cells, such as T cells, and some cancer cells. These checkpoints help keep natural immune responses from being too strong but can often prevent T cells from killing cancer cells which they would do if not for the effect of checkpoint inhibitors. When these checkpoints are blocked, immune cells, such as T cells can kill cancer cells more efficiently.

Checkpoint proteins, such as PD-L1 on tumor cells and PD-1 on T cells, help keep immune responses in check. It is known that the binding of PD-L1 to PD-1 keeps T cells from killing tumor cells in the body. Moreover, blocking the binding of PD-L1 to PD-1 with an immune checkpoint inhibitor (anti-PD-L1 or anti-PD-1) has been shown to allow the T cells to kill tumor cells.

Additionally, checkpoint proteins, such as B7-1/B7-2 on antigen-presenting cells (APC) and CTLA-4 on T cells, help keep the body's immune responses in check. When the T-cell receptor (TCR) binds to antigen and major histocompatibility complex (MHC) proteins on the APC and CD28 binds to B7-1/B7-2 on the APC, the T cell can be activated. However, the binding of B7-1/B7-2 to CTLA-4 has been shown to keep the T cells in the “inactive” state so they are not able to kill tumor cells in the body. But, blocking the binding of B7-1/B7-2 to CTLA-4 with an immune checkpoint inhibitor (e.g., anti-CTLA-4 antibody) has been shown to allow the T cells to be active and to kill tumor cells. Currently, immune checkpoint inhibitors have been approved to treat a variety of cancers, including but not limited to, breast, bladder, cervical, colon, head and neck, liver, lung, stomach, and rectal cancer(s) to name a few.

### (a) KEYTRUDA (Pembrolizumab)

Pembrolizumab (Merck & Co., Rahway, NJ) is an FDA approved immune checkpoint inhibitor. It is a monoclonal antibody that binds to the protein PD-1 on the surface of immune cells called T cells. It works by keeping cancer cells from suppressing the immune system. This allows the immune system to attack and kill the cancer cells. KEYTRUDA is approved in a multitude of cancer indications, including but not limited to, cervical cancer, cSCC, endometrial cancer, esophageal cancer, head and neck squamous cell cancer, kidney cancer (RCC), melanoma, colorectal cancer, stomach cancer, breast cancer, and bladder cancer.

### (b) OPDIVO (Nivolumab)

Nivolumab (Bristol Meyers Squibb, Lawrenceville, NJ) is an FDA approved monoclonal antibody that binds to the protein PD-1 to help immune cells kill cancer cells better and is used to treat many different types of cancer. These include cancers that express the protein PD-L1 or that have certain mutations in genes involved in DNA repair. Nivolumab is used alone or combination with other drugs to treat certain types of classic Hodgkin lymphoma, colorectal cancer, esophageal cancer, gastroesophageal junction cancer, head and neck squamous cell carcinoma, hepatocellular carcinoma, malignant pleural mesothelioma, melanoma, non-small cell lung cancer, renal cell carcinoma, stomach cancer, and urothelial carcinoma (a type of bladder or urinary tract cancer). It is currently being studied in the treatment of additional cancer indications. Novolumab may block PD-1 and help the immune system kill cancer cells. It is a type of immune checkpoint inhibitor.

### (c) YERVOY (Ipilimumab)

Ipilimumab (Bristol Meyers Squibb, Lawrenceville, NJ) is a monoclonal antibody medication that works to activate the immune system by targeting CTLA-4, a protein receptor that downregulates the immune system. Generally, Cytotoxic T lymphocytes (CTLs) can recognize and destroy cancer cells. However, an inhibitory mechanism interrupts this destruction. Ipilimumab turns off this inhibitory mechanism and boosts the body's immune response against cancer cells. Ipilimumab is approved by the FDA in combination with nivolumab in several cancer indications. Notably, In October 2020, the U.S. FDA approved the combination of nivolumab with ipilimumab for the first-line treatment of adults with malignant pleural mesothelioma that cannot be removed by surgery. This is the first drug regimen approved for mesothelioma in sixteen (16) years and the second FDA-approved systemic therapy for mesothelioma. However, a major drawback of ipilimumab therapy is its association with severe and potentially fatal immunological adverse effects due to T cell activation and proliferation, occurring in ten (10) to twenty (20) percent (%) of patients. Serious adverse effects include stomach pain, bloating, constipation, diarrhea, fever, trouble breathing, and urinating problems.

### (d) TENCENTRIQ (Atezolizumab)

Atezolizumab (F. Hoffmann-La Roche Ltd., Basel, CH) is a monoclonal antibody designed to bind with a protein called PD-L1 expressed on tumour cells and tumour-infiltrating immune cells, blocking its interactions with both PD-1 and B7.1 receptors. By inhibiting PD-L1, Atezolizumab enables the activation of T cells. Atezolizumab is approved in the US, EU and countries around the world, either alone or in combination with targeted therapies and/or chemotherapies, for various forms of metastatic NSCLC, small cell lung cancer (SCLC) and hepatocellular carcinoma (HCC), as well as certain types of metastatic urothelial cancer, PD-L1-positive metastatic triple-negative breast cancer and for the treatment of people with BRAF V600 mutation-positive advanced melanoma.

### V.) Immune Checkpoint Specific Antibodies

Another aspect of the invention provides antibodies that bind to immune checkpoints. Briefly, immune checkpoints are proteins on the surface of immune cells that bind to partner proteins on other cells. When the proteins bind, they send an “off” signal to the immune cells, preventing them from attacking. Immune checkpoints are important for self-tolerance, which keeps the immune system from attacking healthy cells. They also help limit the duration and intensity of immune responses to minimize damage to other tissues. Some types of cancers can use immune checkpoints to avoid being attacked by the immune system. Immunotherapy drugs called immune checkpoint inhibitors (ICIs) can help the immune system find and attack cancer cells by blocking the checkpoint proteins. Examples of well-known immune checkpoints are (i) cytotoxic T lymphocyte-associated antigen 4 (CTLA-4), (ii) programed death-1 (PD1), (iii) lymphocyte activation gene-3 (LAG-3), (iv) T-cell immunoglobulin and mucin domain-containing molecule 3 (TIM-3), (v) B and T lymphocyte attenuator (BTLA) and (vi) T-cell immunoreceptor with immunoglobulin and ITIAM domains (TIGIT). The inhibition of immune checkpoints by blocking the co-inhibitory signaling pathways provides a promising immune therapy against tumors.

As is known in the art, antibodies specific to immune checkpoints of the invention are particularly useful in the treatment of cancer, for prognostic assays, imaging, diagnostic, and therapeutic methodologies. In one embodiment, an antibody specific to immune checkpoints binding assay is disclosed herein for use in detection of cancer, for example, in an immunoassay. Similarly, such antibodies specific to immune checkpoints are useful (e.g., when combined with BNCT, in the treatment, and/or prognosis of cancer (for example, head and neck cancer and cancers treated with BNCT) to the extent an immune checkpoint is implicated in these other cancers. Moreover, intracellularly expressed antibodies (e.g., single chain antibodies) are therapeutically useful in treating cancers in which immune checkpoint(s) are involved.

Various methods for the preparation of antibodies, specifically monoclonal antibodies, are well known in the art. For example, antibodies can be prepared by immunizing a suitable mammalian host using antibodies specific to immune checkpoints-related protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of antibodies specific to immune checkpoints can also be used, such as an immune checkpoint-GST-fusion protein. In a particular embodiment, a GST fusion protein comprising all or most of the amino acid sequence of an immune checkpoint is produced, and then used as an immunogen to generate appropriate antibodies. In another embodiment, an immune checkpoint-related protein is synthesized and used as an immunogen.

In addition, naked DNA immunization techniques known in the art are used (with or without purified immune checkpoint-related protein or immune checkpoint expressing cells) to generate an immune response to the encoded immunogen (for review, see DONNELLY et. al., 1997, Ann. Rev. Immunol. 15:617-648).

Preferred methods for the generation of antibodies specific to immune checkpoints are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or another carrier protein. In some circumstances, direct conjugation using, for example, carbodiimide reagents are used; in other instances, linking reagents such as those supplied by Pierce Chemical Co. (Rockford, IL), are effective. Administration of an immune checkpoint immunogen is often conducted by injection over a suitable time period and with use of a suitable adjuvant, as is understood in the art. During the immunization schedule, titers of antibodies can be taken to determine adequacy of antibody formation.

Monoclonal antibodies specific to immune checkpoints can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is an immune checkpoint-related protein. When the appropriate immortalized cell culture is identified, the cells can be expanded, and antibodies produced either from in vitro cultures or from ascites fluid.

The antibodies or fragments of the invention can also be produced by recombinant means. Regions that bind specifically to the desired regions of an immune checkpoint protein can also be produced in the context of chimeric or complementarity-determining region (CDR) grafted antibodies of multiple species origin. Humanized or human antibodies specific to immune checkpoints can also be produced and are preferred for use in therapeutic contexts. Methods for humanizing murine and other non-human antibodies, by substituting one or more of the non-human antibody CDRs for corresponding human antibody sequences, are well known (see for example, JONES et. al., 1986, Nature 321:522-525; RIECHMANN et. al., 1988, Nature 332:323-327; VERHOEYEN et. al., 1988, Science 239:1534-1536). See also, CARTER et. al., 1993, Proc. Natl. Acad. Sci. USA 89:4285 and SIMS et. al., 1993, J. Immunol. 151:2296.

In one embodiment, human monoclonal antibodies specific to immune checkpoints of the invention can be prepared using VelocImmune mice into which genomic sequences bearing endogenous mouse variable segments at the immunoglobulin heavy chain (VH, DH, and JH segments) and/or kappa light chain (VK and JK) loci have been replaced, in whole or in part, with human genomic sequences bearing unrearranged germline variable segments of the human immunoglobulin heavy chain (VH, DH, and JH) and/or kappa light chain (VK and JK) loci (Regeneron, Tarrytown, N.Y.). See, for example, U.S. Pat. Nos. 6,586,251, 6,596,541, 7,105,348, 6,528,313, 6,638,768, and 6,528,314.

In addition, human antibodies of the invention can be generated using the HuMAb mouse (Medarex, Inc.) which contains human immunoglobulin gene Mini loci that encode unrearranged human heavy (mu and gamma) and kappa light chain immunoglobulin sequences, together with targeted mutations that inactivate the endogenous mu and kappa chain loci (see e.g., LONBERG, et. al. (1994) Nature 368 (6474): 856-859).

In another embodiment, fully human antibodies specific to immune checkpoints of the invention can be raised using a mouse that carries human immunoglobulin sequences on transgenes and trans chromosomes, such as a mouse that carries a human heavy chain transgene and a human light chain trans chromosome. Such mice, referred to herein as “KM mice,” such mice are described in TOMIZUKA et. al. (2000) Proc. Natl. Acad. Sci. USA 97:722-727 and PCT Publication WO 02/43478 to TOMIZUKA, et. al.

Human monoclonal antibodies specific to immune checkpoints of the invention can also be prepared using phage or yeast display methods for screening libraries of human immunoglobulin genes. Such phage display methods for isolating human antibodies are established in the art. See for example: U.S. Pat. Nos. 5,223,409; 5,403,484; and 5,571,698 to LADNER et. al.; U.S. Pat. Nos. 5,427,908 and 5,580,717 to DOWER et. al.; U.S. Pat. Nos. 5,969,108 and 6,172,197 to MCCAFFERTY et. al.; and U.S. Pat. Nos. 5,885,793; 6,521,404; 6,544,731; 6,555,313; 6,582,915 and 6,593,081 to GRIFFITHS et. al.

Human monoclonal antibodies specific to immune checkpoints of the invention can also be prepared using SCID mice into which human immune cells have been reconstituted such that a human antibody response can be generated upon immunization. Such mice are described in, for example, U.S. Pat. Nos. 5,476,996 and 5,698,767 to WILSON, et. al.

Additionally, human antibodies specific to immune checkpoints of the present invention can be made with techniques using transgenic mice, inactivated for antibody production, engineered with human heavy and light chains loci referred to as Xenomouse (Amgen Fremont, Inc., formerly Abgenix, Inc.). An exemplary description of preparing transgenic mice that produce human antibodies can be found in U.S. Pat. No. 6,657,103. See, also, U.S. Pat. Nos. 5,569,825; 5,625,126; 5,633,425; 5,661,016; and 5,545,806; and MENDEZ, et. al. Nature Genetics, 15:146-156 (1998); KELLERMAN, S. A. & GREEN, L. L., Curr. Opin. Biotechnol 13, 593-597 (2002).

Any of the methods of production above result in antibodies that have a certain ability to bind immune checkpoint proteins, or homologs or fragments or polypeptide sequences having 85, 90, 91, 92, 93, 94, 95, 96, 9, 98, or 99% sequence identity to any respective immune checkpoint protein.

The binding affinity (K_{D}) of the antibodies, binding fragments thereof, and antibody drug conjugates comprising the same for an immune checkpoint protein may be 1 mM or less, 100 nM or less, 10 nM or less, 2 nM or less or 1 nM or less. Alternatively, the K_{D} may be between 5 and 10 nM; or between 1 and 2 nM. The K_{D} may be between 1 micromolar and 500 micromolar or between 500 micromolar and 1 nM.

The binding affinity of the antigen binding protein is determined by the association constant (Ka) and the dissociation constant (Kd) (K_{D}=Kd/Ka). The binding affinity may be measured by BIACORE for example, by capture of the test antibody onto a protein-A coated sensor surface and flowing immune checkpoint protein over this surface. Alternatively, the binding affinity can be measured by FORTEBIO for example, with the test antibody receptor captured onto a protein-A coated needle and flowing an immune checkpoint protein over this surface. One skilled in the art can identify other suitable assays known in the art to measure binding affinity.

Engineered antibodies of the invention include those in which modifications have been made to framework residues within V_{H} and/or V_{L} (e.g., to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Engineering of the V_{H} and/or V_{L} can also be made to modify the binding affinity to the antigen. For example, changing residues within the frameworks and/or CDR regions to increase affinity, or reduce affinity to an immune checkpoint protein are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by CARR, et. al.

In addition, or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody specific to immune checkpoints of the invention may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by BODMER, et. al. The number of cysteine residues in the hinge region of CH1 is altered to, for example, facilitate assembly of the light and heavy chains or to increase or decrease the stability of the antibodies specific to immune checkpoints.

In another embodiment, the Fc hinge region of an antibody is mutated to decrease the biological half-life of the antibodies specific to immune checkpoints. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by WARD, et. al.

In another embodiment, the antibody specific to immune checkpoints is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by PRESTA et. al.

In another embodiment, the antibodies specific to immune checkpoints comprise anti-PD-1 antibodies.

In another embodiment, the antibodies specific to immune checkpoints comprise anti-PD-L1 antibodies.

In another embodiment, the antibodies specific to immune checkpoints comprise anti-CTLA-4 antibodies. In another embodiment, the antibodies specific to immune checkpoints comprise anti-LAG-3 antibodies.

In another embodiment, the antibodies specific to immune checkpoints are conjugated to a therapeutic agent.

In another embodiment, the antibodies specific to immune checkpoints are conjugated to a borylated compound to create an antibody boron conjugate (ABC).

In another embodiment, the antibodies specific to immune checkpoints are conjugated to a borylated GLUT-1 transporter compound.

In another embodiment, the antibodies specific to immune checkpoints are conjugated to a carborane boron enriched linker (BEL) to create an antibody boron conjugate (ABC).

In another embodiment, the antibodies specific to immune checkpoints are conjugated to a boron enriched linker (BEL) to create an antibody boron conjugate (ABC).

In another embodiment, the antibodies specific to immune checkpoints are used in combination with BNCT.

In another embodiment, the antibodies specific to immune checkpoints are used in combination with BNCT and further in combination with a borylated amino acid compound.

In another embodiment, the antibodies specific to immune checkpoints are used in combination with BNCT and further in combination with a borylated amino acid compound, further comprising BTS.

In another embodiment, the antibodies specific to immune checkpoints are used in combination with BNCT and further in combination with a borylated di-peptide compound.

In another embodiment, the antibodies specific to immune checkpoints are used in combination with BNCT and further in combination with a borylated di-peptide compound, further comprising BPA-BPA.

In another embodiment, the antibodies specific to immune checkpoints are used in combination with BNCT and further in combination with a borylated di-peptide compound, further comprising Ala-BPA.

In another embodiment, the antibodies specific to immune checkpoints are used in combination with BNCT and further in combination with boronophenylalanine (BPA).

Reactivity of the antibodies specific to immune checkpoints can be established by a number of well-known means, including Western blot, immunoprecipitation, ELISA, and FACS analyses using, as appropriate, immune checkpoint-related proteins, immune checkpoint expressing cells or extracts thereof. An antibody or antibodies specific to immune checkpoints or a fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator, a borylated compound, or an enzyme.

One of ordinary skill in the art will appreciate and be enabled to make variations and modifications to the disclosed embodiment without altering the function and purpose of the invention disclosed herein. Such variations and modifications are intended within the scope of the present disclosure.

### VI.) Clinical Design for Immune Checkpoint Inhibitors (ICI) with Neutron Capture Therapy

Antibody-based immune-check point inhibitors are generally given every 3-4 weeks for a period of 2-3 years. This would be a significant contrast with combination therapy with BNCT and ICPs where a single treatment with BNCT may be needed and augmented with only 2-4 applications of ICP antibody before BNCT on the day of BNCT and/or after BNCT with a maximum of 5-10 doses of the ICP antibodies.

Notably, BNCT is usually given as a single fraction, but some patients may receive a second treatment several months after the first, if needed. Under the current standard of care, approximately 25% of patients receive a second treatment of BNCT.

### VII. Methods of Treating Cancer(s) and Other Immunological Disorders Using Immune Checkpoint Inhibitors and BNCT

The identification of an antibody specific to an immune checkpoint protein that is implicated in cancers traditionally treated with BNCT, such as head and neck cancer, opens a number of therapeutic approaches to the treatment of such cancers.

Of note, targeted antitumor therapies have been useful even when the targeted protein is expressed on normal tissues or cells, even vital normal organ tissues. A vital organ is one that is necessary to sustain life, such as the heart or colon. A non-vital organ is one that can be removed whereupon the individual is still able to survive. Examples of non-vital organs are ovary, breast, and prostate.

Accordingly, therapeutic approaches that inhibit the activity of an immune checkpoint protein are useful for patients suffering from cancer where a form of immunotherapy is optimal.

Cancer patients can be evaluated for the presence of ICPs in specific cancer types, preferably using immunohistochemical assessments of tumor tissue, quantitative imaging, or other techniques that reliably indicate the presence and degree of biomarker expression where immune checkpoint inhibitors are an optimal treatment paradigm. Immunohistochemical analysis of tumor biopsies and/or surgical specimens are preferred for this purpose. Methods for immunohistochemical analysis of tumor tissues are well known in the art.

Another aspect of the present disclosure is the use of Boron Neutron Capture Therapy (BNCT) as a treatment modality. Briefly, BNCT is a binary treatment modality in which neither component alone is lethal or toxic to the tumor. The two components comprise (i) the infusion or delivery of a capture compound, which preferentially is concentrated in the tumor, and (ii) the irradiation of the tumor site by neutrons or by protons. In BNCT, given the large cross-section of thermal neutron interactions with ¹⁰B, there is consequently a high probability of a splitting of Boron nucleus into ⁴He²⁺ and ⁷Li⁺. Given that the ionization capability of He²⁺ and Li⁺ is high, and the distances travelled are short, then the cells preferably enriched by Boron are killed and the healthy cells are damaged much less due to the lack of high concentration of boron. Given this, the advantage of BNCT is the destruction of tumor cells without a highly traumatic surgical procedure. However, as will be understood by one of skill in the art, success is predicated high concentration and selective localization of ¹⁰B or another capture agent in tumor cells.

In one embodiment, ¹⁹B or other capture agent is then given to a patient and the ¹⁰B or other capture agent is localized into a tumor cell. The ¹⁰B are concentrated into the tumor and the tumor is irradiated using epithermal neutrons. The tumor cells are destroyed.

a. Combination Therapy

In view of the foregoing, in one embodiment, there is synergy when tumors, including human tumors, are treated with BNCT and antibodies specific for immune checkpoints and/or in conjunction with additional chemotherapeutic agents or radiation or any combination thereof. In other words, the inhibition of tumor growth by BNCT is enhanced more than expected when combined with an immune checkpoint inhibitor as well as chemotherapeutic agents or radiation or combinations thereof. Synergy may be shown, for example, by greater inhibition of tumor growth with combined treatment than would be expected from a treatment of only BNCT or an immune checkpoint inhibitor such as KEYTRUDA, OPDIVO, YERVOY, and/or TENCENTRIQ or the additive effect of treatment with a immune checkpoint inhibitor and a chemotherapeutic agent or radiation. Preferably, synergy is demonstrated by remission of the cancer where remission is not expected from treatment either from BNCT or an immune checkpoint inhibitor or with treatment using an additive combination of an immune checkpoint inhibitor and a chemotherapeutic agent or radiation or an immunotherapy such as CAR-T or NK cell therapy.

The method for inhibiting growth of tumor cells using BNCT and an immune checkpoint inhibitor and/or a combination of chemotherapy or radiation or both comprises administering BNCT in combination with an immune checkpoint inhibitor before, during, or after commencing chemotherapy or radiation therapy, as well as any combination thereof (i.e. before and during, before and after, during and after, or before, during, and after commencing the chemotherapy and/or radiation therapy). For example, the combination of BNCT and an immune checkpoint inhibitor is typically administered between 1 and 60 days, preferably between 3 and 40 days, more preferably between 5 and 12 days before commencing radiation therapy and/or chemotherapy. However, depending on the treatment protocol and the specific patient's needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the chemotherapeutic agent are administered as a separate treatment. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation can be either external or internal to the patient being treated. When the source is external to the patient, the therapy is known as external beam radiation therapy (EBRT). When the source of radiation is internal to the patient, the treatment is called brachytherapy (BT) or radioimmunotherapy (RIT) where antibodies are conjugated with radioisotopes such as Actinium. In one embodiment, the radiation therapy is boron neutron capture therapy (BNCT).

The above-described therapeutic regimens may be further combined with additional cancer treating agents and/or regimes, for example additional chemotherapy, cancer vaccines, signal transduction inhibitors, agents useful in treating abnormal cell growth or cancer, antibodies (e.g. Anti-CTLA-4 antibodies as described in WO/2005/092380 (Pfizer)) or other ligands that inhibit tumor growth by binding to IGF-1R, and cytokines.

When the patient is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3-′-(trifluoromethyl)propionanilide) may be used.

In a preferred embodiment, the combination of BNCT combined with an immune checkpoint inhibitor is performed.

In another embodiment, the combination of BNCT combined with an immune checkpoint inhibitor is performed and further comprises the combination with a borylated amino acid compound.

In another embodiment, the combination of BNCT combined with an immune checkpoint inhibitor is performed and further comprises the combination with a borylated amino acid compound, further comprising BTS.

In another embodiment, the combination of BNCT combined with an immune checkpoint inhibitor is performed and further comprises the combination with a borylated di-peptide compound.

In another embodiment, the combination of BNCT combined with an immune checkpoint inhibitor is performed and further comprises the combination with a borylated di-peptide compound, further comprising BPA-BPA.

In another embodiment, the combination of BNCT combined with an immune checkpoint inhibitor is performed and further comprises the combination with a borylated di-peptide compound, further comprising Ala-BPA.

In another embodiment, the combination of BNCT combined with an immune checkpoint inhibitor is performed and further comprises the combination with a borylated amino acid compound, wherein the immune checkpoint inhibitor is selected from the group consisting of anti-PD-1 antibodies, anti-PD-L1 antibodies, and anti-CTLA-4 antibodies.

In another embodiment, the combination of BNCT combined with an immune checkpoint inhibitor is performed and further comprises the combination with a borylated amino acid compound, wherein the borylated amino acid comprises BTS, and wherein the immune checkpoint inhibitor is selected from the group consisting of anti-PD-1 antibodies, anti-PD-L1 antibodies, anti-CTLA-4, and anti-LAG-3 antibodies

In another embodiment, the combination of BNCT combined with an immune checkpoint inhibitor is performed and further comprises the combination with a borylated di-peptide compound, wherein the immune checkpoint inhibitor is selected from the group consisting of anti-PD-1 antibodies, anti-PD-L1 antibodies, anti-CTLA-4 antibodies, and anti-LAG-3 antibodies.

In another embodiment, the combination of BNCT combined with an immune checkpoint inhibitor is performed and further comprises the combination with a borylated di-peptide compound, wherein the borylated di-peptide compound comprises BPA-BPA, and wherein the immune checkpoint inhibitor is selected from the group consisting of anti-PD-1 antibodies, anti-PD-L1 antibodies, anti-CTLA-4 antibodies, and anti-LAG-3 antibodies.

In another embodiment, the combination of BNCT combined with an immune checkpoint inhibitor is performed and further comprises the combination with boronophenylalanine (BPA).

In a further preferred embodiment, the combination of BNCT combined with KEYTRUDA is performed.

In a further preferred embodiment, the combination of BNCT combined with OPDIVO is performed.

In a further preferred embodiment, the combination of BNCT combined with YERVOY is performed.

In a further preferred embodiment, the combination of BNCT combined with TENCENTRIQ is performed.

In a further preferred embodiment, the combination of BNCT combined with LIBTAYO (cemiplimab) is performed.

In a further preferred embodiment, the combination of BNCT combined with JEMPERELI (dostarlimab) is performed.

In a further preferred embodiment, the combination of BNCT combined with TEVIMBRA (tislelizumab) is performed.

In a further preferred embodiment, the combination of BNCT combined with IMFINZI (durvalumab) is performed.

In a further preferred embodiment, the combination of BNCT combined with BEVENCIO (avelumab) is performed.

In a further preferred embodiment, the combination of BNCT combined with IMJUDO (tremelimumab).

In a further preferred embodiment, the combination of BNCT combined with OPDUALAG (relatlimab) is performed.

The above therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

### VIII.) Kits/Articles of Manufacture

For use in the laboratory, prognostic, prophylactic, diagnostic, and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise a BNCT capture agent that is or can be detectably labeled and/or is loaded with an immune checkpoint inhibitor and/or an antibody specific to an immune checkpoint of the disclosure. Kits can comprise a container comprising a drug unit. The kit can include all or part of the BNCT treatment components and/or an immune checkpoint inhibitor.

The kit of the invention will typically comprise the container described above and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic or laboratory application, and can also indicate directions for either in vivo or in vitro use, such as those described herein. Directions and/or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as a cancer or other immunological disorder.

The terms “kit” and “article of manufacture” can be used as synonyms.

In another embodiment of the invention, an article(s) of manufacture containing compositions used in BNCT as well as an immune checkpoint inhibitors. The article of manufacture typically comprises at least one container and at least one label. Suitable containers include, for example, bottles, vials, syringes, and test tubes. The containers can be formed from a variety of materials such as glass, metal, or plastic. The container can hold a component used in BNCT as well as or in addition to an immune checkpoint inhibitor.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be an immune checkpoint inhibitor or a component used in BNCT or both.

The article of manufacture can further comprise a second container comprising a pharmaceutically acceptable buffer, such as phosphate-buffered saline, Ringer's solution, and/or dextrose solution. It can further include other materials desirable from a commercial and user standpoint, including other buffers, diluents, filters, stirrers, needles, syringes, and/or package inserts with indications and/or instructions for use.

### EXEMPLARY EMBODIMENTS

1) A method of performing Neutron Capture Therapy in the treatment of human cancer comprising:
   a. loading a neutron capture agent onto a delivery vehicle;
   b. injecting the neutron capture agent into a tumor, whereby said neutron capture agent accumulates into a tumor cell; and
   c. irradiating the neutron capture agent with neutrons.
2) The method of claim 1, wherein the irradiation comprises epithermal neutrons.
3) The method of claim 2, wherein the irradiation triggers neutron activation.
4) The method of claim 1, wherein the Neutron Capture Agent is selected from the group consisting of the Boron isotope ¹⁰B, the Lithium isotope ⁶Li, the Cadmium isotope ¹¹³Cd, the Samarium isotope ¹⁴⁹Sm, the Gadolinium isotope ¹⁵⁷Gd, BTS, BTS (OMe), ¹⁰BPA, ¹⁰BPA-BPA, or ¹⁰Ala-BPA.
5) A kit comprising the neutron capture agent of claim 1.
6) A kit comprising the neutron capture agent of claim 5, which is specifically adapted to perform the method of claim 1.
7) A Dosage Unit Form comprising the neutron capture agent of claim 1.
8) The composition of claim 1, whereby the BPMO is surface modified with a phosphonate.
9) The composition of claim 1, whereby the BPMO is surfaced modified with a phosphonate and diol.
10) The method of claim 1, further comprising administering an immune checkpoint inhibitor.
11) The method of claim 10, wherein the immune checkpoint inhibitor comprises an antibody.
12) The method of claim 10, wherein the antibody binds CTLA4.
13) The method of claim 10, wherein the antibody binds PD1/PDL1.
14) The method of claim 10, wherein the immune checkpoint inhibitor is KEYTRUDA.
15) The method of claim 10, wherein the immune checkpoint inhibitor is OPDIVO.
16) The method of claim 10, wherein the immune checkpoint inhibitor is YERVOY.
17) The method of claim 10, wherein the immune checkpoint inhibitor is TENCENTRIQ.
18) The method of claim 18, wherein the immune checkpoint inhibitor is LIBTAYO.
19) The method of claim 18, wherein the immune checkpoint inhibitor is JEMPERELI.
20) The method of claim 18, wherein the immune checkpoint inhibitor is TEVIMBRA.
21) The method of claim 18, wherein the immune checkpoint inhibitor is IMFINZI.
22) The method of claim 18, wherein the immune checkpoint inhibitor is BEVENCIO.
23) The method of claim 18, wherein the immune checkpoint inhibitor is IMJUDO.
24) The method of claim 18, wherein the immune checkpoint inhibitor is OPDUALAG.
25) A method of performing the combination of Neutron Capture Therapy and immunotherapy comprising an immune checkpoint inhibitor in the treatment of human cancer comprising:
   a. loading a neutron capture agent onto a delivery vehicle;
   b. injecting the neutron capture agent into a tumor, whereby said neutron capture agent accumulates into a tumor cell;
   c. irradiating the neutron capture agent with neutrons; and
   d. administering to a patient an immune checkpoint inhibitor.
26) The method of claim 18, wherein the irradiation comprises epithermal neutrons.
27) The method of claim 18, wherein the irradiation triggers neutron activation.
28) The method of claim 18, wherein the immune checkpoint inhibitor comprises an antibody.
29) The method of claim 28, wherein the antibody binds CTLA4.
30) The method of claim 28, wherein the antibody binds PD1/PDL1.
31) The method of claim 28, wherein the immune checkpoint inhibitor is KEYTRUDA.
32) The method of claim 28, wherein the immune checkpoint inhibitor is OPDIVO.
33) The method of claim 28, wherein the immune checkpoint inhibitor is YERVOY.
34) The method of claim 28, wherein the immune checkpoint inhibitor is TENCENTRIQ.
35) The method of claim 28, wherein the immune checkpoint inhibitor is LIBTAYO.
36) The method of claim 28, wherein the immune checkpoint inhibitor is JEMPERELI.
37) The method of claim 28, wherein the immune checkpoint inhibitor is TEVIMBRA.
38) The method of claim 28, wherein the immune checkpoint inhibitor is IMFINZI.
39) The method of claim 28, wherein the immune checkpoint inhibitor is BEVENCIO.
40) The method of claim 28, wherein the immune checkpoint inhibitor is IMJUDO.
41) The method of claim 28, wherein the immune checkpoint inhibitor is OPDUALAG.
42) The method of claim 1, wherein the cancer is selected from the group consisting of breast cancer, brain cancer, gastric cancer, lung cancer, colon cancer, and head and neck cancer.
43) The method of claim 25, wherein the cancer is selected from the group consisting of breast cancer, brain cancer, gastric cancer, lung cancer, colon cancer, and head and neck cancer.
44) The method of claim 1, further combined with radiation therapy.
45) The method of claim 1, further combined with chemotherapy.
46) The method of claim 25, further combined with radiation therapy.
47) The method of claim 25, further combined with chemotherapy.
48) A dosage unit form, comprising a neutron capture agent, whereby the neutron capture agent comprises a borylated amino acid.
49) The dosage unit form of claim 48, wherein the borylated amino acid comprises BTS.
50) A dosage unit form, comprising a neutron capture agent, whereby the neutron capture agent comprises a borylated di-peptide.
51) The dosage unit form of claim 50, wherein the borylated di-peptide comprises BPA-BPA.
52) The dosage unit form of claim 49, wherein the BTS is within a range of 250 mg/kg to 1,500 mg/kg.
53) The dosage unit form of claim 51, wherein the BPA-BPA is within a range of 250 mg/kg to 1,500 mg/kg.

### EXAMPLES

Various aspects of the invention are further described and illustrated by way of the several examples that follow, none of which is intended to limit the scope of the invention.

### Example 1: Experimental Design to Generate Vaccine Effect/Abscopal Effect in Animal Model(s) Using “Hot” CT26 LAT1 Positive Colon Carcinoma Cell Line

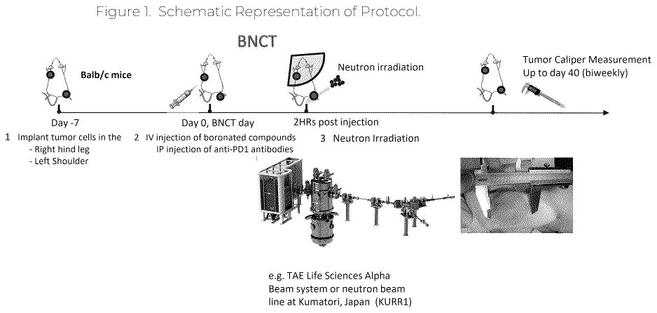

Experiment design was based on the Schematic Representation shown in FIG. 1 as well as the mechanism of abscopal effect at the cellular level (FIG. 2) and the mechanism of immune checkpoint inhibitors (FIG. 3). Notably, “hot” tumors are characterized by a microenvironment infiltrated by T cells, which can attack tumor cells, and often respond well to immunotherapy and ablation therapy such as radiation. Briefly, CT26 LAT1 positive colon carcinoma cell line was implanted in the right leg of BALB/c immune competent mice. When the tumor reaches 300 mm³, the mice were dosed with a specific boronated compound and irradiated for ninety (90) minutes. Following the irradiation, the treated tumors were transplanted to the left leg of the mice to measure the abscopal effect on the tumors. For use as a negative control, untreated mice also had transplanted untreated tumors to the left leg. The tumor growth was monitored at multiple timepoints throughout the protocol.

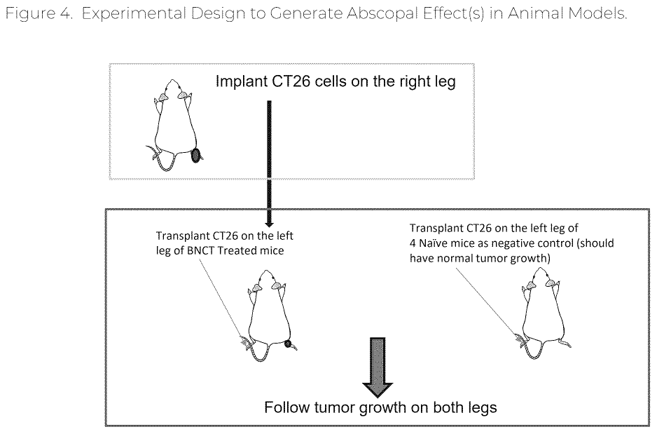

A non-limiting exemplary schematic of the experimental design is shown in FIG. 4.

### Example 2: Abscopal Effect in BNCT Mouse Allogenic Model, In Vivo

In this experiment, the abscopal effect in BNCT using the CT26 allogenic mouse model was tested in vivo using the following protocols. Briefly, the experiments were performed using the design set forth in the example entitled “Experimental Design to Generate Abscopal Effect in Animal Model(s) Using “Hot” CT26 LAT1 positive colon carcinoma cell line”. 900 mg/kg of borylated di-peptide BPA-BPA and 900 mg/kg of borylated di-peptide Ala-BPA. The tumors were grown to 300 mm³ and were irradiated.

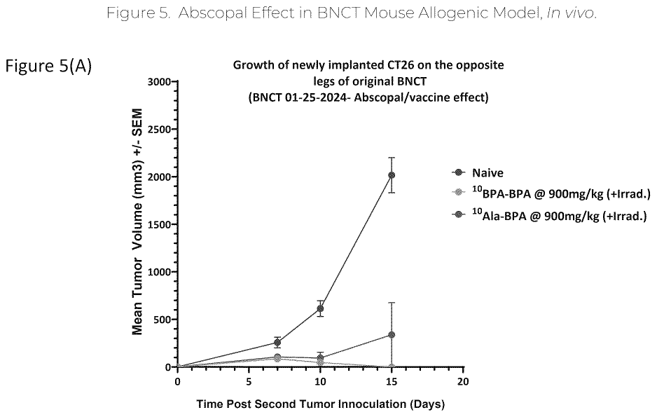

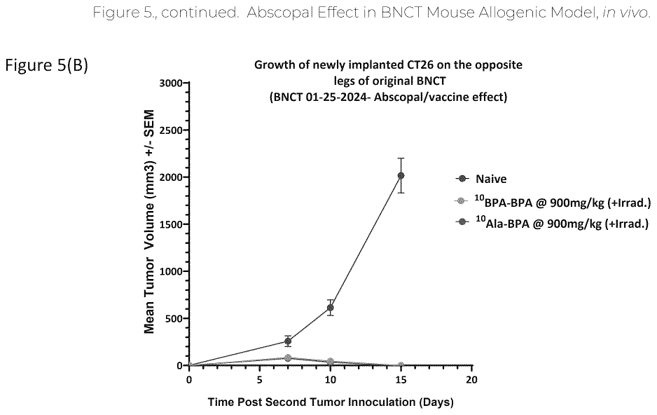

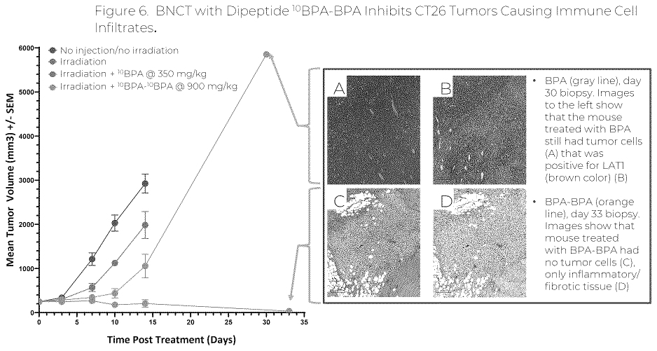

The results show that the abscopal effect (vaccine effect) of BPA-BPA showed significant reduction at day fifteen (15) compared to Ala-BPA and when compared to the control. (See, FIG. 5(a)).

Furthermore, in following with the experimental design, the tumor growth of the newly implanted CT26 tumor on the opposite legs of the original BNCT was measured. The results show that both BPA-BPA and Ala-BPA showed significant abscopal effect at day fifteen (15) compared to the control. (See, FIG. 5(b)).

### Example 3: BNCT with Dipeptide ¹⁰BPA-BPA Inhibits CT26 Tumors Causing Immune Cell Infiltrates, In Vivo

In another experiment, further testing was performed at a later timepoint to determine the amount of tumor. Briefly, the experiments were performed using the design set forth in the example entitled “Experimental Design to Generate Abscopal Effect in Animal Model(s) Using “Hot” CT26 LAT1 positive colon carcinoma cell line”. 900 mg/kg of borylated dipeptide BPA-BPA and 350 mg/kg of boronophenylalanine (BPA) were implanted in Balb-c mice. The tumors were grown to 300 mm³ and were irradiated.

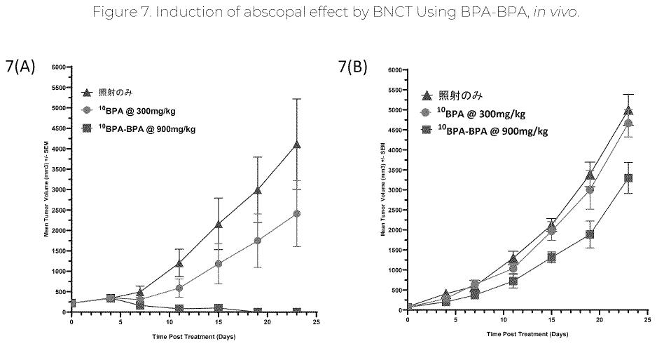

The results show that at day thirty (30) biopsy of the BPA tumors, the mouse treated with BPA still had tumor cells (A) that was positive for LAT1 (brown color) (B). Furthermore, the results show that at day thirty-three (33) biopsy of the BPA-BPA tumors the mouse treated with BPA-BPA had no tumor cells (C), only inflammatory/fibrotic tissue (D). (See, FIG. 6).

### Example 4: Induction of the Abscopal Effect by BNCT Using BPA-BPA in CT26 Tumor Model, In Vivo

In another experiment, further testing was performed to determine the induction of the abscopal effect using borylated di-peptide BPA-BPA. Briefly, the experiments were performed using the design set forth in the example entitled “Experimental Design to Generate Abscopal Effect in Animal Model(s) Using “Hot” CT26 LAT1 positive colon carcinoma cell line”. 900 mg/kg of borylated dipeptide BPA-BPA and 300 mg/kg of boronophenylalanine (BPA) were implanted in Balb-c mice. The tumors were grown to 300 mm³ and were irradiated.

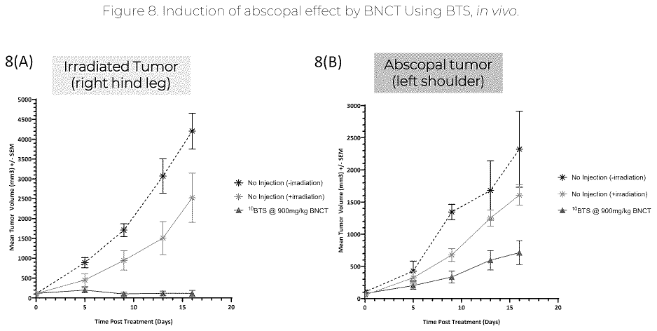

The results show a complete response was observed with BPA-BPA on primary BNCT tumor, whereas only a partial response was achieved with BPA (FIG. 7(A). Additionally, significant growth inhibition of abscopal tumor was observed in mice treated with BPA-BPA compared to no injection (34% less than no injection, p<0.05, day 23; multiple t test) (FIG. 7(B).

### Example 5: Induction of the Abscopal Effect by BNCT Using BTS in CT26 Tumor Model, In Vivo

In another experiment, further testing was performed to determine the induction of the abscopal effect using borylated amino acid BTS. Briefly, the experiments were performed using the design set forth in the example entitled “Experimental Design to Generate Abscopal Effect in Animal Model(s) Using “Hot” CT26 LAT1 positive colon carcinoma cell line”. 900 mg/kg of borylated amino acid BTS were implanted in Balb-c mice. The tumors were grown to 300 mm³ and were irradiated.

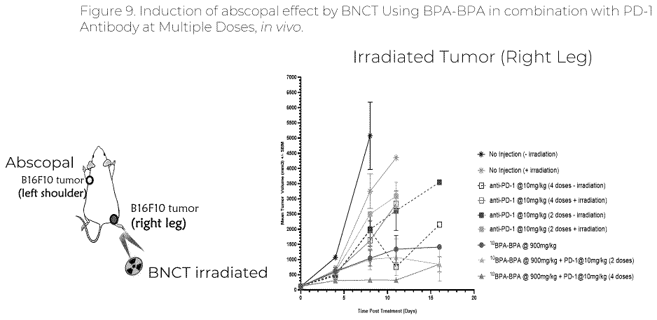

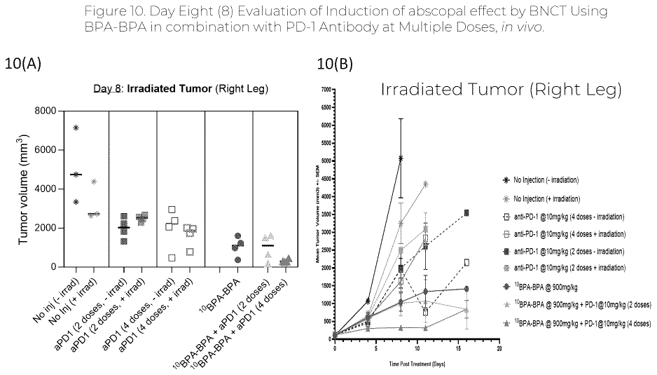

The results show a complete response was observed with BTS on the primary BNCT tumor (FIG. 8(A)) and strong inhibition on the abscopal tumor (FIG. 8(B)).

### Example 6: Experimental Design to Generate Combination Studies and Vaccine Effect/Abscopal Effect in Animal Model(s) Using “Cold” B16F10 Mouse Melanoma Cell Line

Experiment design was based on the Schematic Representation shown in FIG. 1 as well as the mechanism of abscopal effect at the cellular level (FIG. 2) and the mechanism of immune checkpoint inhibitors (FIG. 3). Notably, “cold” tumors are characterized by lacking significant T cell infiltration and are less responsive to immunotherapy and ablation therapy such as radiation. By way of background, B16F10 cells are a subclone of the original B16 melanoma cell line, selected through multiple rounds of injection and harvesting of metastatic cells, making them more invasive than the parent or B16-F1 lines. Additionally, B16F10 tumors are highly metastatic under certain conditions. They are known to metastasize to various organs, including the lungs and liver. From a scientific point of view, the importance of using B16F10 in the experimental design is important for several reasons. First, B16F10 is a syngeneic melanoma model frequently used in mouse pre-clinical studies. Second, Cutaneous melanoma is the sixth most common type of cancer overall in humans, and its incidence has been increasing yearly for the last 30 years. Third, if cutaneous melanoma is diagnosed at an early stage (T-I, N-0, M-0) the tumor can be removed surgically in most cases leading to a cure (The 5-yr. survival rate for early-stage cutaneous melanoma is as high as 97%). Fourth, if the melanoma is not diagnosed early, then patients may already have metastasis to regional or distant sites at the time of diagnosis (at least 13% of melanoma patients already have metastatic tumors at the time of diagnosis). Fifth, metastasis is a significant problem in the treatment of cancer, accounting for more than 90% of cancer-related deaths in people. Finally, treatment options for metastatic melanoma are limited because metastatic melanoma is resistant to most traditional cancer therapies.

Materials: The Following Materials were Developed and Used in these Experiments:
RMP1-14 Antibody. An anti-mPD1 antibody in combination BNCT experiments (part of check point inhibitor therapy or check point blockade (CPB) (Schematic Below)

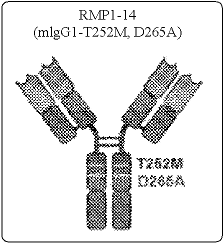

Checkpoint proteins. Such as PD-L1 on tumor cells and PD-1 on T cells, help keep immune responses in check, binding of PD-L1 to PD-1 can impact tumor cell recognition by the immune system and protect them from being killed by T cells. Blocking the binding of PD-L1 to PD-1 with an immune checkpoint inhibitor such as the anti-PD1 antibody, KEYTRUDA (Merck, Rahway, New Jersey).
Reactor. KURR1 Reactor (Kyoto University, Kyoto, Japan), Six (6) minutes, 5 MW, twelve (12) mice per holder.

Briefly, combination studies whereby immune checkpoint inhibitors (anti-PD1) with BNCT testing tumor effect and abscopal effect was performed using B10F16 in C57BL6 immune competent mice. BPA-BPA was given i.v. at 900 mg/kg. RMP1-14 (anti-mouse PD1) was given intra-peritoneally (i.p.) at 10 mg/kg on day negative four (−4), zero (0), day four (4) and day eight (8) or at 10 mg/kg on day (−)zero (−0) and day zero (0) (4 doses versus 2 doses). When the tumor reaches 300 mm³, the mice were dosed with a specific boronated compound and irradiated for six (6) minutes. Following the irradiation, the treated tumors were transplanted to the left shoulder of the mice to measure the abscopal effect on the tumors. For use as a negative control, untreated mice also had transplanted untreated tumors to the left shoulder. The tumor growth was monitored at multiple timepoints throughout the protocol depending on the specific assay being measured.

### Example 7: Induction of the Abscopal Effect by BNCT Using BPA-BPA in Combination with PD-1 Antibody at Multiple Doses, In Vivo

In another experiment, further testing was performed to determine the induction of the abscopal effect using borylated di-peptide BPA-BPA in combination with PD-1 antibody. Briefly, the experiments were performed using the design set forth in the example entitled “Experimental Design to Generate Combination Studies and Vaccine Effect/Abscopal Effect in Animal Model(s) Using “Cold” B16F10 mouse melanoma cell line.” Notably, 900 mg/kg of borylated di-peptide BPA-BPA, 10 mg/kg of anti-PD-1 antibody (at multiple doses with and without irradiation), and a combination of 900 mg/kg of BPA-BPA along with 10 mg/kg of anti-PD-1 antibody at multiple doses were implanted in C57BL6 mice and tested per the protocol.

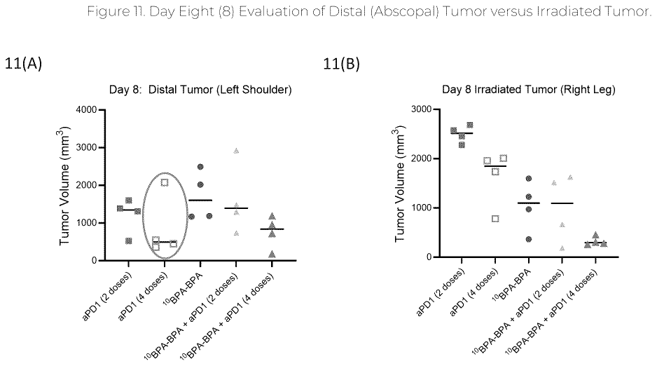

The results show that the anti-PD1 Mab plus BPA-BPA combo was most effective at suppressing tumor growth of both the treated tumor and abscopal tumor. Additionally, the combination improved overall mouse survival rate (FIG. 9). Notably, mouse mortality was high in the B16F10 model due to aggressive growth characteristics and propensity to metastasize to the lungs. Thus, this is likely a significant cause of mouse mortality.

### Example 8: Day Eight (8) Snapshot Assessment of Induction of the Abscopal Effect by BNCT Using BPA-BPA in Combination with PD-1 Antibody at Multiple Doses, In Vivo

In another analysis of the experiment previously discussed (See, Example 7), a day eight (8) snapshot of the results were assessed.

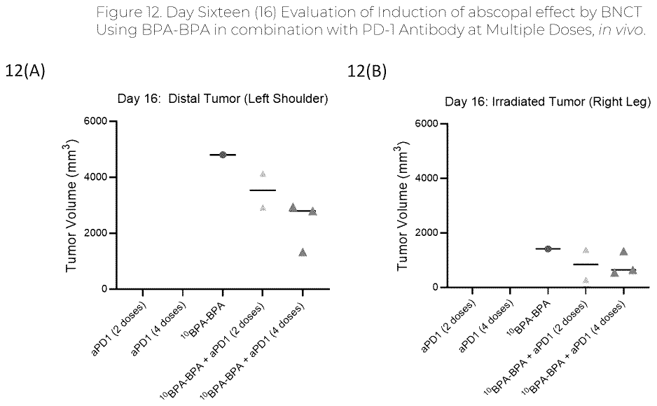

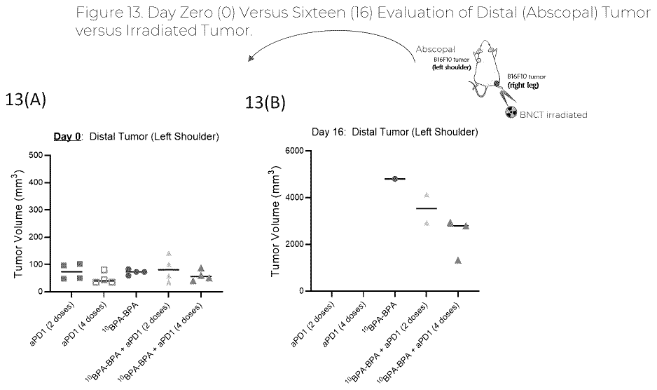

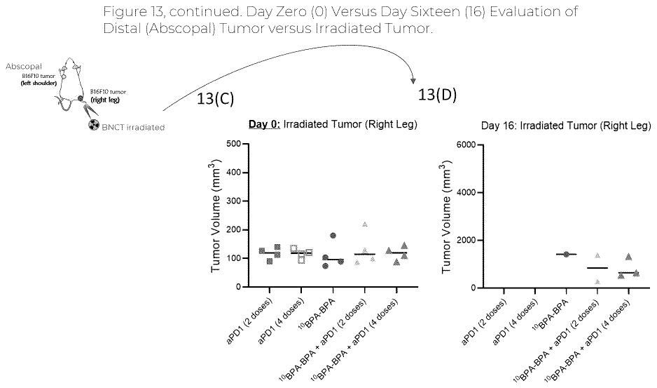

The results show that the anti-PD1 Mab (with 4 doses) plus BPA-BPA combo was the most effective treatment. (See, FIG. 10). Additionally, when comparing the distal (abscopal) tumor to the irradiated tumor, a synergistic effect of anti-PD1 Mab (with 4 doses) plus BPA-BPA is observed in the distal tumor (circled) (FIG. 11(A) when compared to the irradiated tumor (FIG. 11(B)).

### Example 9: Day Sixteen (16) (End of Experiment) Snapshot Assessment of Induction of the Abscopal Effect by BNCT Using BPA-BPA in Combination with PD-1 Antibody at Multiple Doses, In Vivo

In another analysis of the experiment previously discussed (See, Example 7), an end of study day sixteen (16) snapshots of the results was assessed.

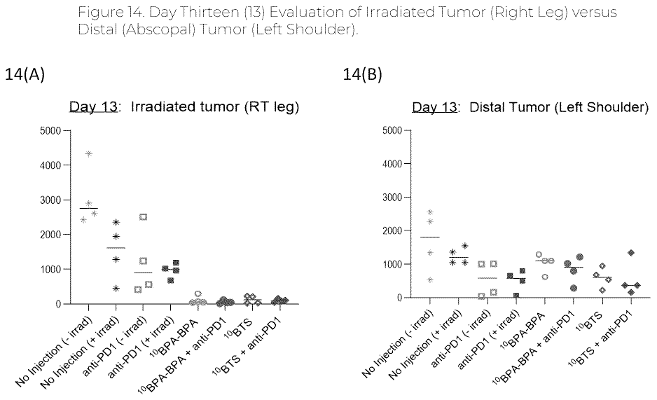

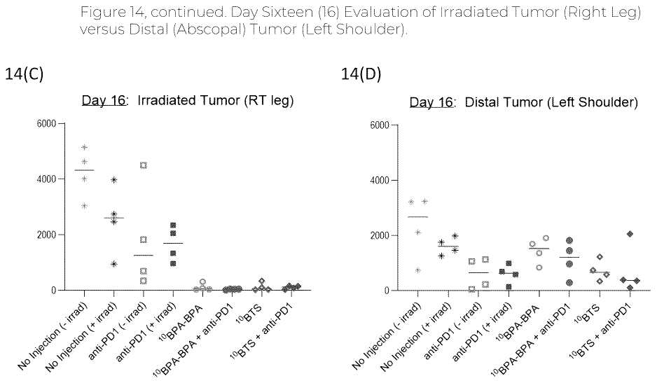

The results show that the anti-PD1 Mab (with 4 doses) plus BPA-BPA combo was the most effective treatment and enhances the survival rate in a dose-dependent manner. The irradiated tumor size is smaller than the distal tumors on day 16 (See, FIGS. 12(A) and 12(B)). Additionally, when comparing the distal (abscopal) tumor to the irradiated tumor from day zero (0) to day sixteen (16), it is clear that the anti-PD1 Mab (with 4 doses) plus BPA-BPA enhances overall survival rate. The distal tumor is smaller than the irradiated tumor on day zero (0). Furthermore, the distal tumor was significantly larger when compared to the irradiated tumor on day sixteen (16). These results show the combination of BNCT with the anti-PD1 Mab (with 4 doses) plus BPA-BPA reduces tumor growth and enhances overall survival rate in mice. (See, FIGS. 13(A), 13(B), 13(C), and 13(D).

### Example 10: Day Thirteen (13), Day Sixteen (16), and Day Twenty-Four (24) Snapshot Assessment of Induction of the Abscopal Effect by BNCT Using BPA-BPA and BTS in Combination with PD-1 Antibody at Multiple Doses, In Vivo

In another analysis of the experiment previously discussed (See, Example 1), an assessment of multiple timepoints (day 13, day 16, and day 24) was performed and the results were assessed. Briefly, 7-weeks-old Balb/c female mice were purchased (CLEA Japan, Inc.). CT26 cells were grown in RPMI medium supplemented with 10% FBS and 1% Penicillin/Streptomycin and cultured with 5% humidified CO₂. Seven (7) days prior to BNCT, CT26 cells were subcutaneously transplanted into two sites—1e6 cells in right leg and 0.5e6 cells in left shoulder. BTS and BPA-BPA were intravenously administered into tumor-grafted mice. After dosing, mice were placed in mouse restrainers on a neutron irradiation rack. The mice were then irradiated at the Kyoto University (Kyoto Japan) reactor for 6 minutes with thermal neutron operating power of 5 MW. After the irradiation, tumor diameters were measured using calipers, and the tumor growth and body weights were monitored every 3-4 days up to day 40. The mice were treated in compliance with the recommendations for the Handling of Laboratory Animals for Biomedical Research compiled by the Committee on Ethical Handling Regulations for Laboratory Animal Experiments, Kyoto University and euthanized according to animal care protocol.

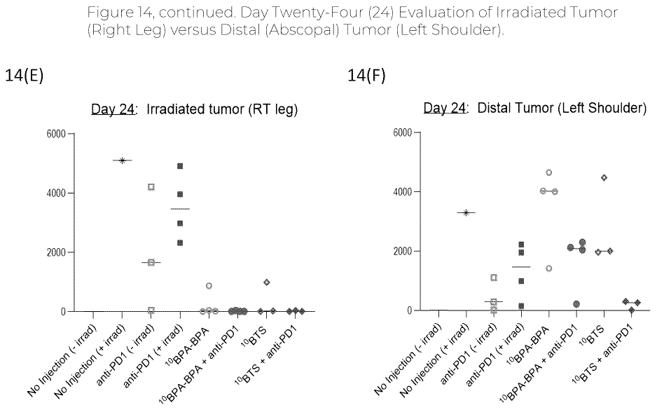

The results show that the anti-PD1 Mab plus BTS and the anti-PD1 Mab plus BPA-BPA combo was the most effective treatment and enhances the survival rate in a dose-dependent manner. The irradiated tumor size is smaller than the distal tumors on day 13 (See, FIGS. 14(A) and 14(B)). Additionally, when comparing the distal (abscopal) tumor to the irradiated tumor from day sixteen (16), it is clear that the anti-PD1 Mab plus BPA-BPA and the anti-PD1 plus BTS enhances overall survival rate. The irradiated tumor is smaller than the distal tumor on day sixteen (16). (See, FIGS. 14(C) and 14(D)). However, it is notable that the anti-PD1 plus BTS shows signs of enhanced abscopal effect at day sixteen (16) versus day thirteen (13). Furthermore, when comparing the distal (abscopal) tumor to the irradiated tumor from day twenty-four (24), it is clear that the anti-PD1 Mab plus BPA-BPA and the anti-PD1 plus BTS enhances overall survival rate. The irradiated tumor is smaller than the distal tumor on day twenty-four (24). (See, FIGS. 14(E) and 14(F)). Notably, the distal tumor was significantly larger when compared to the irradiated tumor on day twenty-four (24) and when compared to BPA-BPA plus anti-PD1. However, the combination of BTS plus anti-PD1 shows continued enhanced abscopal effect (especially when compared to day 13 and day 16). These results show the combination of BNCT with the anti-PD1 Mab plus BPA-BPA and anti-PD1 plus BTS reduces tumor growth and enhances overall survival rate in mice. (See, FIGS. 14(A), 14(B), 14(C), 14(D), 14(E), and 13(F)).

### Example 11: Human Clinical Trials for the Treatment of Human Carcinomas Through the Combination of BNCT and Immune Checkpoint Inhibitors

The combination therapy of BNCT and immune checkpoint inhibitors (ICIs) are used in accordance with the present invention which specifically accumulates in a tumor cell and are used in the treatment of certain tumors and other immunological disorders and/or other diseases. In connection with each of these indications, two clinical approaches are successfully pursued.
I.) Adjunctive therapy: In adjunctive therapy, patients are treated with a combination of BNCT and an ICI also in combination with a chemotherapeutic or pharmaceutical or biopharmaceutical agent or a combination thereof. Primary cancer targets are treated under standard protocols by the addition BNCT and then irradiated and then treated with an ICI. Protocol designs address effectiveness as assessed by the following examples, including but not limited to, reduction in tumor mass of primary or metastatic lesions, increased progression free survival, overall survival, improvement of patients health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.
II.) Monotherapy: In connection with the use of the combination of BNCT and an ICI as a monotherapy of tumors, the BNCT and ICI are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patients health, disease stabilization, as well as the ability to reduce usual doses of standard chemotherapy and other biologic agents.

### Dosage

Dosage regimens may be adjusted to provide the optimum desired response. For example, a single BNCT treatment may be administered with one ICI, or several BNCT treatments and several divided doses of an ICI may be administered over time, or the dose may be proportionally reduced or increased as indicated by the exigencies of the therapeutic situation. “Dosage Unit Form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the BNCT treatment (for example, the BNCT capture agent) as well as the specific characteristics of the ICI, the individual mechanics of the irradiation mechanism (reactor) and the particular therapeutic or prophylactic effect to be achieved, and (b) the limitations inherent in the art of compounding such an capture agent for the treatment of sensitivity in individuals.

### Clinical Development Plan (CDP)

The CDP follows and develops treatments of using capture agents combined with ICIs which are then irradiated using Neutron Capture Therapy in connection with adjunctive therapy or monotherapy. Trials initially demonstrate safety and thereafter confirm efficacy in repeat doses. Trials are open label comparing standard chemotherapy with standard therapy plus ICIs and capture agents which are then irradiated using Boron Neutron Capture Therapy. As will be appreciated, one non-limiting criterion that can be utilized in connection with enrollment of patients is concentration of a capture agent in a tumor in the BNCT paradigm as well as the likelihood a specific ICI is deemed useful/necessary as determined by standard detection methods known in the art.

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

[symbol]

1. A method of performing the combination of Neutron Capture Therapy and immunotherapy, whereby the immunotherapy comprises an immune checkpoint inhibitor (ICI) in the treatment of cancer comprising:
a. loading a neutron capture agent onto a delivery vehicle;
b. injecting the neutron capture agent into a tumor, whereby said neutron capture agent accumulates into a tumor cell;
c. irradiating the neutron capture agent with neutrons; and
d. administering to a patient an immune checkpoint inhibitor.

2. The method of claim 1, wherein the irradiation triggers neutron activation.

3. The method of claim 1, wherein the immune checkpoint inhibitor comprises an antibody.

4. The method of claim 3, wherein the antibody binds CTLA-4.

5. The method of claim 3, wherein the antibody binds PD1.

6. The method of claim 3, wherein the antibody binds PD-L1.

7. The method of claim 1, wherein the immune checkpoint inhibitor is KEYTRUDA.

8. The method of claim 1, wherein the immune checkpoint inhibitor is OPDIVO.

9. The method of claim 1, wherein the immune checkpoint inhibitor is YERVOY.

10. The method of claim 1, wherein the immune checkpoint inhibitor is TENCENTRIQ.

11. The method of claim 1, wherein the neutron capture agent is a borylated amino acid compound.

12. The method of claim 1, wherein the neutron capture agent is BTS.

13. The method of claim 1, wherein the neutron capture agent is BTS (Ome).

14. The method of claim 1, wherein the neutron capture agent is a borylated di-peptide compound.

15. The method of claim 1, wherein the neutron capture agent is BPA-BPA.

16. The method of claim 1, wherein the neutron capture agent is ¹⁰BPA.

17. The method of claim 1, wherein the cancer is selected from the group consisting of breast cancer, brain cancer, gastric cancer, lung cancer, colon cancer, and head and neck cancer.

18. The method of claim 1, wherein the neutron capture therapy comprises boron neutron capture therapy (BNCT).

19. A dosage unit form, comprising a neutron capture agent and an immune checkpoint inhibitor.

20. A kit comprising the dosage unit form of claim 19.
